(19) United States
(12) Patent Application Publication
(10) Pub. No.: US 2006/0149208 A1
(43) Pub. Date: Jul. 6, 2006

(54) Absorbent article with elastomeric end regions

(75) Inventor: James M. Carr, Kaukauna, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/026,218

(22) Filed: Dec. 30, 2004

(51) Int. Cl. A61F 13/15

(52) U.S. Cl. 604/385.22; 604/385.3

(57) ABSTRACT

Absorbent articles having elastomeric end regions and methods of making such articles are disclosed. The absorbent article includes a bodyside liner and a garment-side outer cover. In particular embodiments, the liner includes a base layer of material and at least two strips or regions of elastomeric material attached to the base layer material with a space therebetween such that a center region of the base layer material is bordered on at least two end by composite regions of the elastomeric materials and the base layer material. In particular embodiments, the base layer material comprises a neckable non-extensible material. In other embodiments, the base layer material comprises an inherently extensible material. In certain embodiments, the composite region or regions can form a portion of the outer cover. Absorbent articles employing the material can have stretchable or elastomeric end regions.

### TECHNICAL FIELD OF THE INVENTION

The present invention relates generally to the field of absorbent articles and garments, such as children's training pants, disposable diapers, incontinence articles, and the like, and more particularly to an improved product design and a process to make such improved product designs.

### BACKGROUND

Many types of disposable absorbent articles such as disposable diapers, training pants, feminine care articles, incontinence articles, and the like, utilize a design incorporating an absorbent pad, a surge layer, a bodyside liner material, containment flaps, a liquid impervious barrier layer, and side portions that can be brought together to close the article around the wearer's body. Often, various portions of these product designs are elasticized in some way to provide a comfortable fit and a gasket function to help reduce leakage. The liquid transfer and absorbency capabilities of the absorbent system depend, in large part, on maintaining the structural integrity and characteristics of the component parts. The structure (e.g., bulk weight, density, capillary structure) of the underlying absorbent material is tailored for particular flow rates and total absorbency depending on the type of absorbent article.

The absorbency, fit, and leakage protection properties of these products are determined in large part by the capillary structure of the components making up the absorbent system, and the elastic properties of various materials used in the total construction. The capillary structure of the various absorbent components is specifically designed and it is desirable to maintain the structure during the entire time the product is being used. Currently, many types of products utilize a piecemeal approach to provide elastic properties by attaching elastic or extensible materials to other components that have little or no elastic properties. The overall effect is to provide stretch for gasketing, fit, and comfort in some portions of the product, while keeping the absorbent components in a relatively non-stretched state to maintain the capillary structure for good absorbency. With products where the entire chassis may be stretchable, the stretching of the liner and rest of the absorbent system causes the capillary structure and fluid handling properties to also change. The capillary structure would change if the absorbent components are stretched. For example, if a necked material with a given fiber and capillary structure is used as a bodyside liner material and is stretched in a direction, the fibers are forced to move and/or rotate to accommodate the stretch. This movement and/or rotation of the fibers changes the capillary structure of the necked nonwoven material. If the necked, non-stretched nonwoven had an ideal capillary structure before stretching, the stretched material may no longer have that ideal structure. In general, any changes in the dimensions of the material in width, length, or thickness will change the capillary structure.

A product design that includes elastic materials attached to non-elastic, non-extensible materials often requires a process that brings the various materials together in a rather complicated fashion, and may attach the components together in ways that negate or “tie-up” the functionality of the elastic materials in these areas of attachment. Other attachment means may reduce the functionality of the elastic components, or require more expensive components to overcome the effect of attachment to the article.

It has been found that overall extendable or elastomeric absorbent products are highly desirable for fit, comfort, and containment. It can be seen that a problem may occur in that for optimum absorbency, the product should not extend, but for fit, comfort, and containment, it should extend without the complications that arise from attaching multiple elastic and non-extensible components together. In particular embodiments, the present invention resolves this dilemma.

### SUMMARY OF THE INVENTION

The present invention relates in part to a product design particularly suited for use as an absorbent article, such as disposable diapers, child's training pants, incontinence articles, feminine care products, diaper pants, disposable swim pants, and the like. The product design makes use of a composite material that can comprise many portions of such absorbent articles, such as the bodyside liner material, waist containment dams, and the outer barrier or cover layer. The invention also relates in part to methods for making the improved product design. The invention can greatly reduce the number of different materials required to construct the article, which simplifies the process to produce it, and greatly reduces the number of attachment points, which can hinder the overall performance of the elastic portions of the product.

In one aspect, the invention relates to disposable absorbent articles. In one embodiment, the disposable absorbent article comprises a chassis which defines front and back waist edges, opposite side edges extending between the front and back waist edges, a front waist region adjacent the front waist edge, a back waist region adjacent the back waist edge, a center region positioned longitudinally between the front and back waist regions, a longitudinal direction extending between and generally perpendicular to the front and back waist edges, and a lateral direction generally perpendicular to the longitudinal direction. The article includes a garment-side outer cover, a bodyside liner; and an absorbent body sandwiched between the outer cover and the liner. The liner comprises a base layer of fluid permeable material, and further comprises a first strip of elastomeric material attached to the base layer material in the front waist region to define a first composite region and a second strip of elastomeric material attached to the base layer in the back waist region and longitudinally spaced from the first strip of elastomeric material to define a second composite region, the liner generally overlaying the absorbent body in the center region. The liner is substantially non-elastomeric in the center region, and the liner is stretchable in the longitudinal direction in the front and back waist regions.

In another aspect, the invention relates to cross-machine direction methods of producing such disposable absorbent articles. In one embodiment, the method includes providing a web of substantially non-extensible material, the web extending in a machine direction and defining opposite web side edges; applying a tensioning force to the web in a first direction, causing the web to neck in a second direction perpendicular to the first direction; superimposing and attaching a first strip of elastomeric material along a first side region of the necked web during application of the tensioning force to define a first composite region; superimposing and attaching a second strip of elastomeric material along a second side region of the necked web during application of the tensioning force, the second strip being laterally spaced apart from the first strip, such that the first and second strips define a center region of the web therebetween; attaching the web to an absorbent body; and cutting individual absorbent articles from the web along a cut line which extends in the cross-machine direction, wherein the cut line defines side regions of the absorbent articles, and wherein the first and second side regions of the web respectively define first and second waist regions of the articles, each absorbent article defining a longitudinal direction generally parallel to the cut line and a lateral direction generally perpendicular to the longitudinal direction, wherein each composite region is elastomeric in the longitudinal direction.

In another embodiment, the method includes providing a web of inherently extensible material, the web extending in a machine direction and defining opposite web side edges, wherein the extensible material is extendable in a first direction to at least about 125% of an original, unextended dimension without fracture of the extensible material; superimposing and attaching a first strip of elastomeric material along a first side region of the web while the web is substantially untensioned to define a first composite region; superimposing and attaching a second strip of elastomeric material along a second side region of the web while the web is substantially untensioned, the second strip being laterally spaced apart from the first strip, such that the first and second strips define a center region of the web therebetween; attaching the web while in a substantially non-extended condition to an absorbent body; and cutting individual absorbent articles from the web along a cut line which extends in the cross-machine direction, wherein the lateral cut line defines side regions of the absorbent articles, and wherein the first and second side regions of the web respectively define first and second waist regions of the article, each absorbent article defining a longitudinal direction generally parallel to the lateral cut line, and a lateral direction generally perpendicular to the longitudinal dimension, wherein each composite region is elastomeric in the longitudinal direction.

Aspects of the invention will be described below in greater detail with reference to embodiments shown in the figures.

### BRIEF DESCRIPTION OF THE FIGURES

FIG. 1 is a schematic representation of an exemplary process for forming absorbent articles in accordance with the invention.

FIG. 1A is a cross-sectional view of the material taken along the lines indicated in FIG. 1.

FIGS. 2A, 2B, and 2C are simplified plan views of exemplary composite material in accordance with the invention.

FIG. 3 is a perspective view of an absorbent article that may incorporate the composite material of the invention.

FIG. 4 is a bodyside plan view of an absorbent article that may incorporate the composite material of the invention, with portions cut away to show underlying features.

FIG. 5A is a partial schematic cross-sectional view of the article of FIG. 4 taken along the lines indicated, with certain components removed for clarity.

FIG. 6 is a schematic cross-sectional view of an alternative absorbent article incorporating the composite material of the invention.

FIG. 7 is a schematic cross-sectional view of an alternate embodiment of an absorbent article according to the invention.

FIG. 8 is a schematic cross-sectional view of still another embodiment of an absorbent article according to the invention.

FIG. 9 is a schematic cross-sectional view of an alternate embodiment of an absorbent article according to the invention.

### DETAILED DESCRIPTION OF PARTICULAR EMBODIMENTS

The invention will now be described in detail with reference to particular embodiments thereof. The embodiments are provided by way of explanation of the invention, and are not meant as a limitation of the invention. For example, features described or illustrated as part of one embodiment may be used with another embodiment to yield still a further embodiment. It is intended that the present invention include these and other modifications and variations as come within the scope and spirit of the invention.

Within the context of the present description, the following terms have the following meanings:

“Attached” and “joined” refers to the bonding, adhering, connecting, and any other method for attaching or joining two elements. Two elements will be considered to be attached or joined together when they are bonded directly to one another or indirectly to one another, such as when each is directly attached to an intermediate element.

“Extendable” or “extensible” means that property of a material or composite by virtue of which it stretches or extends in the direction of an applied biasing force by at least about 25% of its relaxed length. An extendable material does not necessarily have recovery properties. For example, an elastomeric material is an extendable material having recovery properties. A meltblown web may be extendable, but not have recovery properties and, thus, be an extensible but non-elastic material. “Non-extensible” refers to a material that does not stretch or extend by at least about 25% of its relaxed length without fracture upon application of a biasing force. Materials that are extensible or elastomeric are not considered “non-extensible.”

“Elastomeric,” “elastic,” and “elasticized” refer to a material or composite which can be elongated by at least 25% of its relaxed length and which will recover, upon release of the applied force, at least 10% of its elongation. It is generally preferred that the elastomeric material or composite be capable of being elongated by at least 100%, more preferably by at least 300%, of its relaxed length and recover at least 50% of its elongation. An elastomeric material is an extendable material having recovery properties.

“Inherently extensible” or “inherently extendable” means a material that is extensible or extendable without having been treated or processed in a way that would impart extensibility to an otherwise non-extensible material. A meltblown web may be inherently extensible without other mechanical manipulation such as necking, but not have recovery properties. Such material would thus be an inherently extensible but non-elastic material.

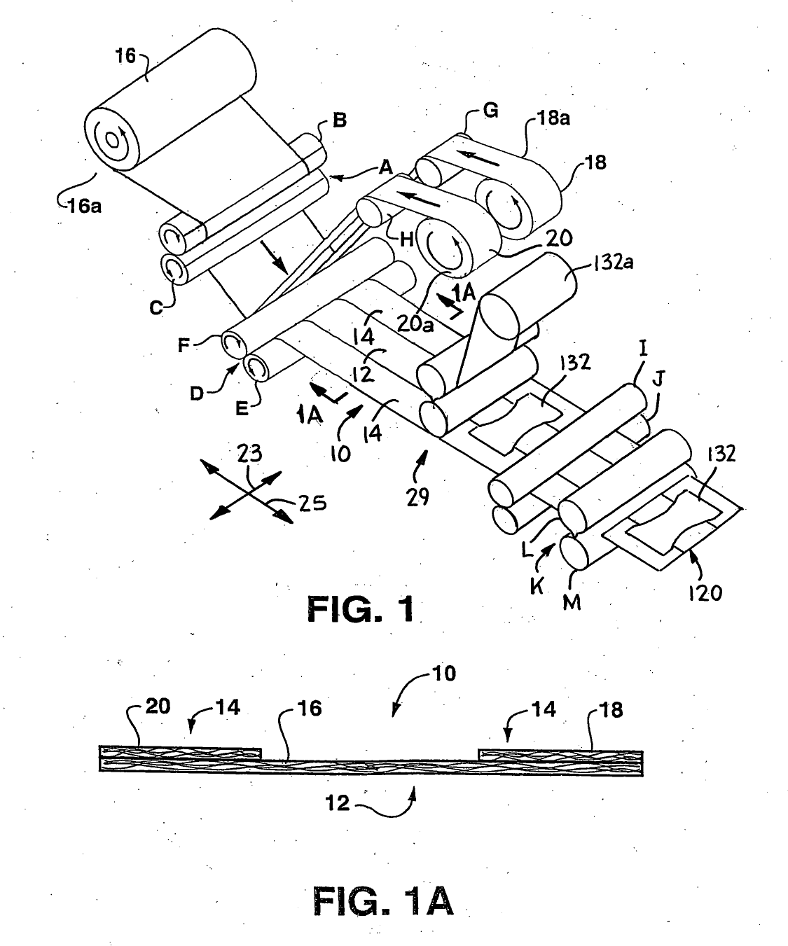

“Machine direction” refers to the length of a fabric or material in the direction in which it is produced or converted, representatively illustrated by reference numeral 25 in FIG. 1, as opposed to “cross direction” or “cross-machine direction” which refers to the width of a fabric in a direction generally perpendicular to the machine direction, representatively illustrated by reference numeral 23 in FIG. 1.

The phrase “machine direction assembly” refers to a manufacturing process in which disposable products travel in an end-to-end (i.e., waist-to-waist) orientation, in the longitudinal direction. In contrast, the phrase “cross-machine direction assembly” refers to a process in which the products travel in a side-by-side orientation such as that representatively illustrated in FIG. 1.

“Member” when used in the singular can refer to a single element or a plurality of elements.

“Necked material” refers to any material which has been constricted in at least one dimension by processes such as, for example, drawing.

“Neck-bonded laminate” refers to a composite material having an elastic member that is bonded to a member while the member is extended in the machine direction creating a necked composite material that is elastic in the cross-direction. Examples of neck-bonded laminates are disclosed in U.S. Pat. Nos. 4,965,122; 4,981,747; 5,226,992; and 5,336,545, which are incorporated herein by reference in their entirety for all purposes.

“Neck stretch-bonded laminate” refers to a laminate made from the combination of a neck-bonded laminate and a stretch-bonded laminate. Examples of necked stretch bonded laminates are disclosed in U.S. Pat. Nos. 5,114,781 and 5,116,662, which are incorporated herein in their entirety by reference thereto for all purposes. A necked stretch bonded laminate can be stretchable in both the machine and cross-machine directions.

“Nonwoven web” refers a web that has a structure of individual fibers or threads which are interlaid, but not in an identifiable, repeating manner. Nonwoven webs may be formed, for example, by a variety of processes including melt-blowing, spunbonding, and bonded carded web processes.

“Reversibly-necked material” refers to a necked material that has been treated while necked to impart memory to the material so that when force is applied to extend the material to it pre-necked dimensions, the necked and treated portions will generally recover to their necked dimensions upon termination of the force. A reversibly-necked material may include more than one layer, such as, multiple layers of spunbonded web, multiple layers of meltblown web, multiple layers of bonded carded web or any other suitable combination of mixtures thereof. The production of reversibly-necked materials is described in U.S. Pat. Nos. 4,965,122 and 4,981,747, incorporated herein by reference for all purposes.

“Stretch-bonded laminate” refers to a composite material having at least two layers in which one layer is a gatherable layer and the other layer is an elastic layer. The layers are joined together when the elastic layer is in an extended condition so that upon relaxing the layers, the gatherable layer is gathered. For example, one elastic member can be bonded to another member while the elastic member is extended at least about 25% of its relaxed length. Such a multilayer composite elastic material may be stretched until the non-extensible layer is fully extended. Examples of stretch-bonded laminates are disclosed, for example, in U.S. Pat. Nos. 4,720,415, 4,789,699, 4781,966, 4,657,802, and 4,655,760, which are incorporated herein by reference in their entirety for all purposes.

“Sheet” refers to a layer which may be either a film, a form, or a nonwoven web.

“Untensioned” as used herein to describe a material web means that the web is under substantially no tension, but does not mean that the web is lacking all tension. In order to handle and process moving webs, some moderate amount of tension is needed to hold the web or material in place. An “untensioned” web or material, as used herein, is under enough tension to process the material, but less than that required to cause substantial deformation of the material.

Various aspects and embodiments of the invention will be described in the context of a material for disposable absorbent articles, such as disposable diapers, children's training pants, incontinence articles, feminine care products, diaper pants, disposable swim pants, and the like. It should be appreciated that this is for illustrative purposes only, and that the invention is not limited to any particular absorbent article, or absorbent articles in general. The material according to the invention may have beneficial uses in any number of applications, such as protective medical clothing, drapes, gowns, and the like.

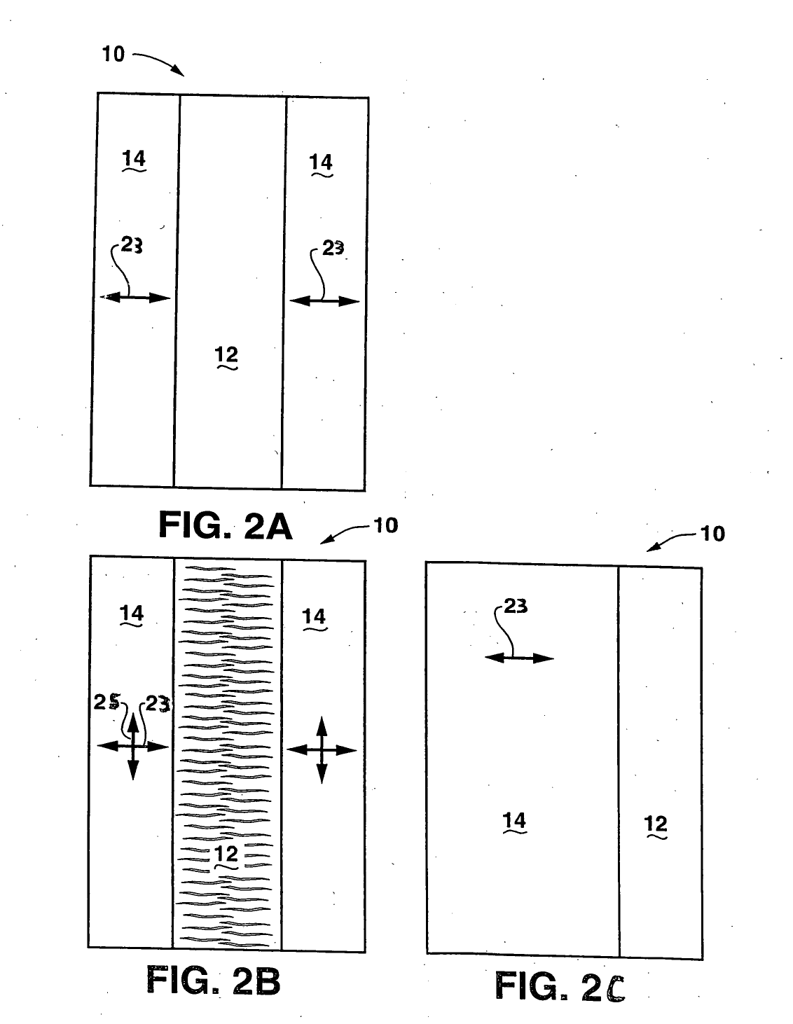

Referring to FIGS. 1, 1A, 2A, 2B, and 2C a composite material 10 according to the invention and method of making are illustrated. The depicted method is related in certain aspects to the method described in U.S. Pat. No. 5,226,992 for making an elastic neck bonded laminate, and the '992 patent is incorporated herein in its entirety for all purposes.

In particular embodiments of the process aspect of the present invention, a material 10 is produced using a base material 16. In particular embodiments, the base material 16 is a generally non-extensible, neckable material. In other embodiments, the base material 16 is an inherently extensible material. Each of these shall now be discussed in turn.

In one particular process of making the material 10 (FIG. 1), a neckable generally non-extensible material 16 is unwound from a supply roll 16a and travels in the direction illustrated by the arrows. The non-extensible material 16 passes through the nip A of the drive roller arrangement formed by rollers B and C. Embodiments of particular types of neckable non-extensible material 16 are described in detail below.

At least a first sheet of elastomeric material 18, such as an elastic film or meltblown, is unwound from a supply roll 18a in the direction indicated by the arrows. In a particular embodiment, a second sheet of elastomeric material 20, such as an elastic film, is unwound from a supply roll 20a in the direction indicated by the arrows. The sheets 18 and 20 can each have a width that is less than that of the non-extensible material 16 after it is necked. For example, the sheets 18 and 20 may have a width that is one-third of the width of the sheet 16. As described below, the respective-widths of the sheets may be varied according to the final use of the composite material 10. Also, the sheets 18 and 20 may have different widths.

The elastomeric sheets 18 and 20 may be the same type of elastomeric material, such as the same film, or a composite of different materials. Particular embodiments of suitable elastomeric materials are described in detail below.

The elastomeric sheets 18 and 20 are directed by guide rollers G and H through the nip D of the bonder roller arrangement formed by rollers E and F. The sheets 18 and 20 can, but need not necessarily, be registered with the material 16 so as to be disposed on and aligned with respective lateral sides of the material 16, as generally indicated in the figures.

The generally non-extensible material 16 passes through the nip A of the S-roller arrangement of rollers B and C in a reverse-S path, and then through the pressure nip D of the bonder rollers E and F. In particular embodiments, material 16 is necked prior to being attached to elastomeric sheets 18 and 20. For example, as shown in FIG. 1, the material 16 may be necked between the supply roll 16a and rollers B and C of the S-roll arrangement, by controlling the peripheral linear speed of the supply roll 16a to be less than the peripheral linear speed of the rollers B and C. The material 16 is thus tensioned in the machine direction 25 and necked-in in the cross direction 23 between the supply roll and the S-roll arrangement. Alternately, the peripheral linear speed of rollers B and C of the S-roll arrangement may be controlled to be less than the peripheral linear speed of the rollers E and F of the bonder roller arrangement, causing the material 16 to be tensioned in the machine direction 25 and necked in the cross direction 23 between the rollers B and C of the S-roll arrangement and rollers E and F of the bonder roll arrangement. By adjusting the difference in speeds of the rollers, the material 16 is tensioned so that it necks a desired amount and is maintained in such tensioned, necked condition while the elastomeric sheets 18 and 20 are attached to the material 16 during their passage through the bonder rollers F and E to form the composite material 10 having composite elastomeric necked bonded laminate strips or regions 14 bordering a center non-elastomeric strip or center region 12.

The material 16 with attached elastomeric sheets 18 and 20 may be kept in a tensioned state throughout its processing so as to maintain the necked condition of the material 16 between the elastomeric sheets 18 and 20. This may be controlled, for example, with downstream rollers I and J. Alternately, the material 16 may be relaxed and re-tensioned throughout its processing so long as it is, in particular embodiments, tensioned during attachment of the elastomeric sheets 18 and 20. The necked material 16b with attached elastomeric sheets 18 and 20 may be wound into a roll while under tension, and the roll may be stored for subsequent use in an in-line manufacturing process. Alternatively, in certain embodiments as representatively illustrated in FIG. 1, the material is directly conveyed in its necked state through an in-line absorbent article manufacturing process wherein it can be adhered to an absorbent body 132 while in the necked state. In such a process, the absorbent body 132 can be provided by methods known in the art, such as by being unwound from a supply roll 132a of absorbent material, and subsequently cut and placed onto the composite material 10 by way of, for example, a variable-speed transfer roll or a vacuum slip-and-cut unit 29. The combination of the composite material 10 and absorbent body 132 can then be cut at cutting point K between cutter L and anvil roll M to any desired size and shape to in part define an absorbent article chassis 120 (outer cover not shown).

The bonder roller arrangement may include a smooth calender roller F and a smooth anvil roller E, or may include a patterned calender roller, such as a pin embossing roller, arranged with a smooth anvil roller. One or both of the calender roller and the smooth anvil roller may be heated and the pressure between these two rollers may be adjusted by well-known means to provide the desired temperature and bonding pressure to join the material 16 to the elastomeric sheets 18 and 20. Alternately, the elastomeric sheets 18, 20 may be attached to the necked material 16 by use of an adhesive, such as an elastomeric adhesive, as are known in the art.

In an alternate embodiment, the material 16 is wound into the supply roll 16a, and subsequently unwound into the present process for joining to the sheets 18 and 20 to produce the composite material 10. Furthermore, the invention is not limited to tensioning the material 16 in the machine direction, as other methods of tensioning the material 16 are contemplated. For example, tenter frames or other cross-machine direction stretcher arrangements that expand the neckable material 16 in other directions, such as the cross-machine direction, may be used.

The necked material 16 and elastomeric sheets 18 and 20 may be completely bonded together and still provide composite elastomeric neck bonded strips 14 with good stretch properties. Alternatively, a bonding pattern, such as a sinusoidal bonding pattern, may be used.

The necked material 16 may be attached to the elastomeric sheets 18 and 20 at least at two places by any suitable means such as, for example, thermal bonding or ultrasonic welding. Joining may be produced by applying heat and/or pressure to the overlaid elastomeric sheets 18 and 20 and the necked material 16 by heating the overlaid portions to at least the softening temperature of the material with the lowest softening temperature to form a reasonably strong and permanent bond between the re-solidified softened portions of the sheets 18 and 20 and material 16. For a given combination of materials, the processing conditions necessary to achieve a satisfactory bonding can be readily determined by one of skill in the art.

The relation between the original dimensions of the neckable material 16 to its dimensions after tensioning determine the approximate limits of stretch of the composite strips 14. For example, referring to FIG. 2A wherein the composite strips 14 are stretchable in the cross-machine direction 23, if the strips have a width of, for example, 10 cm, and it is desired that each of the strips 14 be stretchable to 150% of their width (i.e., to 15 cm), then the original width of the underlying base material 16 along the strips 14 is at least 15 cm. As should readily be understood, the elastic limit of the sheets 18 and 20 need only be as great as the maximum desired elastic limit of the composite strips 14. In other words, the elastic sheets should be able to take the necked material back to its non-necked cross-direction dimensions.

It should be understood that the process described above with respect to FIG. 1 is presented for illustrative purposes only. Other conventional methods and machinery may be readily employed to produce a composite material 10 according to the invention. For example, a tensioned wind-up process may be used to join a necked material 16 and pressure sensitive elastomeric adhesive web of meltblown fibers 18 and 20. In an alternate embodiment, an elastomeric web sheet may be meltblown directly onto the material 16 in the regions corresponding to the composite strips 14. An additional material may be overlaid on the meltblown sheet.

It should also be understood that the composite strips or regions 14 and intermediate strip 12 are not limited to any particular number of material layers. For example, the underlying material 16 may include various combinations of woven or non-woven layers to achieve desired characteristics of the final composite material 10 depending on the particular end use of the material. Likewise, the elastomeric sheets 18 and 20 may include various combinations of materials to provide the strips 14 with desired characteristics.

Referring to FIG. 2A, it can be seen that the material 10 includes the strip 12 of necked material bordered on cross-direction sides by the composite strips 14. The strips 14 are stretchable in the transverse cross-direction 23. This embodiment may be formed, for example, by joining the elastomeric material sheets 18 and 20 in an unstretched state to the necked material 16 (FIGS. 1 and 1A). The center portion 12 is allowed to return to its un-necked dimension (width) upon release of the necking tension. In an alternate embodiment, depicted in FIG. 2B, the strips 14 are stretchable in both the cross-direction 23 and the machine direction 25. This embodiment may be formed, for example, by attaching the elastomeric strips 18 and 20 in a stretched state to the necked material 16. In this way, upon releasing the tensioning force on the necked material 16 and the stretching force on the elastomeric strips 18 and 20, the composite strips 14 essentially become necked stretch-bonded laminates that are stretchable in the machine direction 25 and cross-direction 23, and the center strip 12 is caused to gather in the machine direction 25 and is extendable at least in the machine direction 25 as a result. On the other hand, if the center strip 12 is attached to the absorbent body 132 when the elastic strips 14 are still stretched, gathering of the center strip 12 will not occur and the desired capillary structure of strip 12 is retained.

In an alternate embodiment, the underlying material 16 may be mechanically processed, such as by creping, prior to attachment of the elastic strips. With this embodiment, the composite material 10 would be stretchable in the machine and cross-directions.

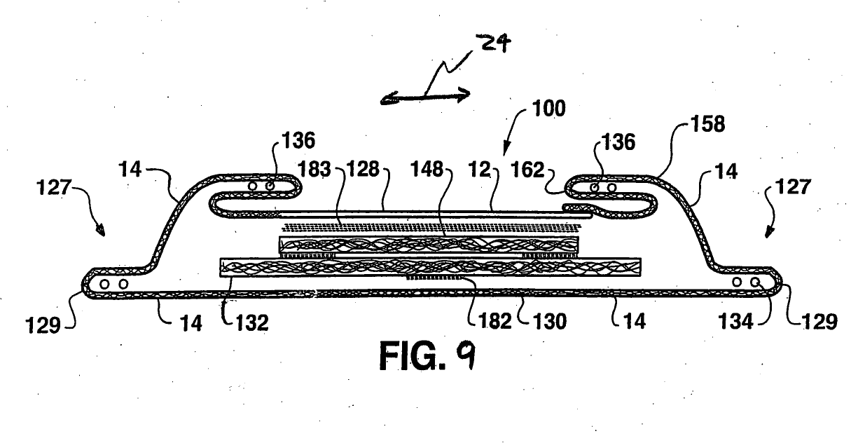

In the embodiment of FIG. 2C, a single composite strip 14 borders a non-extensible region 12. As described in greater detail below, the material 10 of this embodiment may be used in various article configurations, such as the article configurations of FIGS. 8 and 9, described below.

In still another embodiment (not shown), it may be desired to completely border or “frame” the center region 12 with composite strips 14. This configuration would tend to “set” or hold the necked state of the material 16 in the center region 12 such that it has a greater bulk weight. This may prove desirable in that the region 12 may function as a surge layer when overlying an absorbent body, such a surge layer designed primarily to receive, temporarily store, and/or transport liquid along a mutually facing surface with an absorbent body, thereby maximizing the absorbent capacity of the absorbent body. In one embodiment of the “framed” configuration, the composite strips or regions 14 may be stretchable in the machine and cross directions.

The non-extensible material 16 may be any one or a combination of suitable materials that are capable of being necked and attached to an elastomeric material. The non-extensible material 16 may be, for example, any conventional liquid permeable material used as an “inner cover” or bodyside liner of a disposable diaper, training pant, incontinence article, and the like. The material may be a non-porous material that has been perforated to render it breathable. In this regard, the material desirably presents a body-facing surface which is compliant, soft-feeling, and non-irritating to the wearer's skin. Further, the material 16 may be less hydrophilic than an underlying absorbent body of an absorbent article of which it forms a part, and sufficiently porous to be liquid permeable, permitting liquid to readily penetrate through its thickness to reach the absorbent body. A suitable non-extensible material may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers.

Various woven and nonwoven fabrics can be used as the non-extensible material 16. For example, the material may include a meltblown web, a spunbonded web, or a bonded-carded-web composed of synthetic continuous or discrete polymer fibers and/or natural fibers, a pattern bonded spunbonded web, airlaid web, or bonded carded web, as well as combinations thereof. The various fabrics can be composed of natural fibers, synthetic fibers or combinations thereof. In particular aspects, the material may be comprised of polymer fibers, networks, laminates, liquid permeable films, cellulosic fibers, rayon, water swellable gels, as well as combinations thereof. Suitable polymers can include polypropylene, polyethylene, polyester, and bicomponent materials composed of these polyolefins.

The non-extensible material 16 may be composed of a substantially hydrophobic material, and the hydrophobic material may optionally be treated with a surfactant or otherwise processed to impart a desired level of wettability and hydrophilicity. In a particular embodiment of the invention, the material can be a nonwoven, spunbond polypropylene fabric. The fabric can be surface treated with an operative amount of surfactant, such as about 0.6% AHCOVEL Base N62 surfactant, available from ICI Americas, a business having offices located in New Castle, Del. The surfactant can be applied by any conventional means, such as spraying, dipping, printing, brush coating or the like. The fibers forming the nonwoven material may be mono-component, bi-component, or multi-component fibers, and combinations thereof.

The non-extensible material 16 may include blends or laminates of fibers, scrim, webs, and films with perforations, apertures, creping, heat activation, embossing, micro-straining, chemical treatment, or the like, as well as combinations thereof.

The elastomeric materials 18 and 20 may be any one or combination of materials that are capable of being attached to the necked material to provide a desired degree of stretch to the resulting fabric. Depending on the end use of the material, the elastomeric materials 18 and 20 may be breathable and liquid impermeable or liquid resistant. Generally, any suitable elastomeric fiber-forming resin or resin blend may be utilized for nonwoven webs of elastomeric fibers suitable for use as the elastomeric material strips. Likewise, any suitable elastomerid film-forming resin or resin blend may be utilized for elastomeric films suitable for use as the elastomeric material strips. Suitable elastomeric materials can include elastic strands, LYCRA® elastics, elastic films, nonwoven elastic webs, meltblown or spunbond elastomeric fibrous webs, as well as combinations thereof. Examples of elastomeric materials include ESTANE® elastomeric polyurethanes (available from Noveon, Inc. located in Cleveland, Ohio, U.S.A.), PEBAX® elastomers (available from Arkema located in Paris, France), HYTREL® elastomeric polyester (available from E. I. DuPont de Nemours located in Wilmington, Del., U.S.A.), KRATON® elastomer (available from Kraton Polymers located in Houston, Tex., U.S.A.), strands of LYCRA® elastomer (available from Invista of Wichita, Kans., U.S.A.), or the like, as well as combinations thereof.

The elastomeric materials 18 and 20 may be a pressure sensitive elastomer adhesive sheet. For example, the elastomeric material itself may be tacky or, alternatively, a compatible tackifying resin may be added to the extrudable elastomeric compositions described above to provide an elastomeric sheet that can act as a pressure sensitive adhesive, such that the elastomeric sheet can be bonded to a tensioned, necked non-extensible material. In regard to the tackifying resins and tackified extrudable elastomeric composition's, reference is made to the resins and compositions described in U.S. Pat. No. 4,789,699, incorporated herein by reference in its entirety for all purposes.

Any tackifier resin can be used which is compatible with the elastomeric polymer and can withstand the high processing (e.g., extrusion) temperatures. If blending materials such as, for example, polyolefins or extending oils are used, the tackifier resin should also be compatible with those blending materials. Generally, hydrogenated hydrocarbon resins are preferred tackifying resins because of their better stability.

The elastomeric materials 18 and 20 may also be a multilayer material of, for example, two or more individual coherent webs or films. Additionally, the sheets may be a multilayer material in which one or more of the layers contain a mixture of elastic and non-extensible fibers or particulates. An example of this type of material is described in U.S. Pat. No. 4,209,563, incorporated herein in its entirety by reference for all purposes, in which elastomeric and non-elastomeric fibers are commingled to form a single coherent web of randomly dispersed fibers. Another example of such a composite web is disclosed in U.S. Pat. No. 4,100,324, also incorporated herein by reference for all purposes.

In an alternative embodiment, again referring to FIG. 1, the invention relates to a process similar in many respects to the process described above in which material 16 is a non-extensible material. However, in the alternative embodiment which shall now be described, material 16 is an inherently extensible material. The inherently extensible material 16 is unwound from a supply roll 16a and travels in the direction illustrated by the arrows. The extensible material 16 may pass through the nip of various roller arrangements in its course of travel, such as nip A of the drive rollers B, C and the nip D of the bonder rollers E, F, without being tensioned, stretched, necked, or otherwise deformed. Embodiments of particular types of inherently extensible material 16 are described in detail below.

In such an embodiment, the sheets of elastomeric material 18 and 20 are attached to the untensioned extensible material 16. The material 16 with attached elastomeric sheets 18 and 20 is kept in an untensioned state throughout its processing so as to generally maintain its original dimensions in the machine direction and cross direction between the elastomeric sheets 18 and 20. The peripheral speed of the roller pairs B, C and E, F, including optionally downstream rollers I, J, is closely controlled to maintain the untensioned state of the material 16. The composite material 10 may be wound into a roll and stored for subsequent use in an in-line manufacturing process. Alternatively, with a particular manufacturing embodiment, the composite material 10 is subsequently conveyed to an in-line absorbent article manufacturing process wherein it is adhered to an absorbent body 132 in an untensioned state. In such a process, the absorbent body 132 can be provided by methods known in the art, such as by being unwound from a supply roll 132a of absorbent material, and subsequently cut and placed onto the composite material 10 by way of, for example, a variable-speed transfer roll or a vacuum slip-and-cut unit 29. The combination of the composite material 10 and the absorbent body 132 can then be cut at cutting point K between cutter L and anvil roll M to any desired size and shape to in part define an absorbent article chassis 120 (outer cover not shown).

Referring to FIGS. 1, 1A, and 2A-2C to illustrate such an embodiment, a composite material 10 is formed, for example, by joining the elastomeric material sheets 18 and 20 in an untensioned state to the untensioned extensible material 16 (FIGS. 1 and 1A). The resulting composite material 10 is thus a zoned CD elastomeric material. The center strip 12 remains inherently extensible in the cross-direction 23 and the strips 14 are essentially elastomeric-bonded laminates stretchable in the cross-direction. Upon release of a stretching force applied to the composite strips 14, the elastomeric property of the sheets 18, 20 will result in the strips 14 recovering towards their untensioned dimensions. In the final product form of an absorbent article, the strip 12 may be made non-extensible by attaching it to a non-extensible material, such as a non-extensible absorbent body. It should be appreciated that the degree of extensibility of the composite strips 14 will also be a function of the elastomeric property of the sheets 18, 20. For example, referring to FIG. 2A, the composite strips 14 are stretchable in the cross-direction 22 to the extent permitted by the elastomeric sheets 18, 20. Also, the sheets 18, 20 may be extensible to a far greater degree than that of the base material 16, in which the extensibility of the strips 14 is limited by fracture of the material 16. As should readily be understood, the elastic limit of the sheets 18 and 20 need only be as great as the maximum desired extensibility limit of the composite strips 14.

Referring again to FIGS. 1 and 1A, in an alternate embodiment, the elastomeric material strips 18, 20 may be attached in a tensioned state to a base material 16 that is inherently extensible in the machine direction 25 and the cross direction 23. After attachment, the strips 18, 20 are released from tension. Referring to FIG. 2B, the resulting composite material 10 will have an extensible center strip 12. The composite strips 14 are elastomeric in the CD and MD directions. The composite material 10 is thus a zoned CD and MD elastomeric material.

Materials suitable for use as the inherently extensible base material 16 include crimped bicomponent nonwoven materials made from polymers such as meltblown that can contain Kraton® styrenic block copolymers available from Kraton Polymers, Houston, Tex., U.S.A., and metallocene catalyzed olefins or blends, as well as polyethylene, polypropylene, nylon, polyester, and the like.

As described, the composite material 10 may be incorporated for use in a wide variety of absorbent articles, such as disposable diapers, child's training pants, incontinence articles, feminine care products, and the like. The material is particularly suited for use as a bodyside liner material. Exemplary embodiments of absorbent articles will be generally described herein. However, it should be appreciated that the invention is not limited to the described embodiments. The construction and materials used in conventional absorbent articles vary widely and are well known to those of skill in the art. A detailed explanation of every such material and construction is not necessary for purposes of describing the present invention.

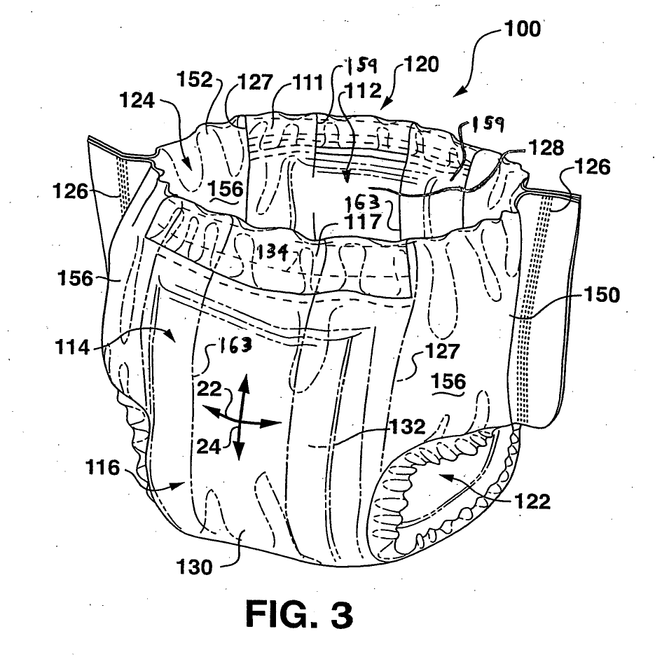

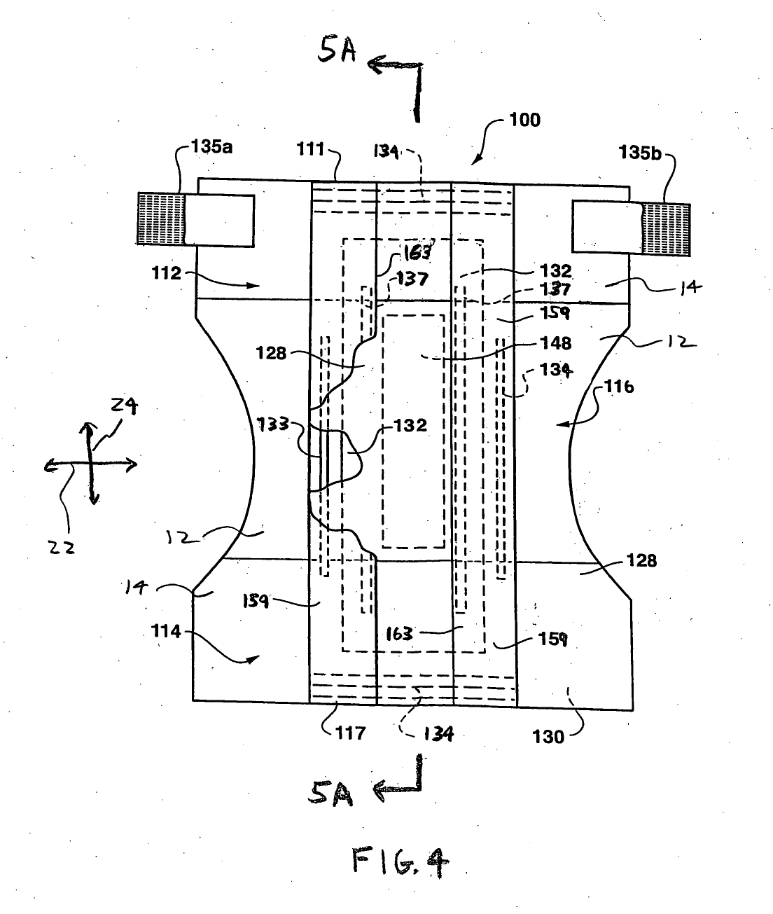

With reference to FIG. 3 in general, an article, such as the representatively shown child's training pant 100, is illustrated. This pant 100 is similar in construction and materials to HUGGIES® PULL-UPS® disposable training pants from Kimberly-Clark Corporation. The article 100 includes a body or chassis 120 having a lengthwise, longitudinal direction 24, a lateral, transverse direction 22, a front waist region 114, a back waist region 112, and an intermediate crotch region 116 interconnecting the front and back waist regions. The waist regions 112 and 114 comprise those portions of the article 100 which when worn, wholly or partially cover or encircle the waist or mid-lower torso of the wearer. In particular configurations, the front 114 and back 112 waist regions may include elastic front and back waistband portions 117, 111 incorporating elastic members 134. In the embodiment of FIG. 3, the elastic waistband portions 111,117 extend only partially across their respective waist regions. In an alternate embodiment, the waistband portions 117, 111 may be generally continuous around the waist opening of the article. The intermediate crotch region 116 lies between and interconnects the waist regions 114 and 112, and comprises that portion of the article 100 which, when worn, is positioned between the legs of the wearer and covers the lower torso of the wearer. Thus, the intermediate crotch region 116 is an area where repeated fluid insults typically occur in the training pant or other disposable absorbent article.

The article 100 includes a substantially liquid-impermeable outer cover member 130, an at least partially liquid-permeable bodyside liner 128, and an absorbent body 132 sandwiched between the outer cover member 130 and the bodyside liner layer 128. The absorbent body may be secured to the outer cover member 130 by an adhesive. The adhesive may be applied along the longitudinally centerline of the absorbent body in the case of a lateral/transverse stretch outer cover, or in a transverse line in the case of a longitudinal stretch outer cover, on in a spot pattern in the case of a lateral and longitudinal stretch outer cover.

For various reasons such as product comfort, performance, size range, etc., it is generally known that particular portions and components of the chassis 120 may be formed of elastomeric materials and thus be stretchable in one or more directions. In the illustrated embodiment of the article 100, the chassis 120 includes stretchable front side panel portions 150 and back side panel portions 152 laterally extending from the central portion of the chassis 120. This configuration is common for training pants and can provide the article with a desired degree of stretchability.

With a known conventional arrangement as depicted in FIG. 3, the panel portions 150, 152 are defined by generally elastomeric side panels 156 that are attached to the lateral sides of the chassis 120 at the waist regions 112, 114, such as along adhesive seam lines 127. The elastomeric side panels may be permanently bonded to the lateral sides of the chassis using attachment means known to those skilled in the art, such as adhesive, thermal or ultrasonic bonding. Particular examples of suitable constructions for securing a pair of elastically stretchable members to the lateral, side portions of an article to extend laterally outward beyond the laterally opposed side regions of the outer cover and liner components of an article can be found in U.S. Pat. No. 4,938,753, which is incorporated by reference herein in its entirety for all purposes. The lateral outboard sides of the side panels 156 may then be permanently or releasably attached along side seams 126 to define a pant structure. These bonded side seams may be tearable as discussed above. Alternately, the side panels may be releasably attachable along the side seams 126 using any type of suitable releasable fastener system, as discussed above. Suitable elastic materials for the side panels 156, as well as a described process of incorporating elastic side panels into a training pant, are described, for example, in the following U.S. Pat. Nos. 4,940,464; 5,224,405; 5,104,116; 5,046,272; and WO 01/88245, all of which are incorporated herein by reference in their entirety for all purposes. In particular embodiments, the elastic material comprises a stretch-thermal laminate (STL), a neck-bonded laminate (NBL), a reversibly necked laminate, or a stretch-bonded laminate (SBL) material. Methods of making such materials are described, for example, in U.S. Pat. Nos. 4,663,220; 5,226,992; and the EP Application 0 217 032, all of which are incorporated herein by reference in their entirety for all purposes.

In an alternate embodiment, the separate panel portions 150, 152 may not be needed, and may be defined by an extension of the chassis 120, for example, extensions of the outer cover member 130, bodyside liner 128, or both. For example, the chassis may include an elastomeric cover member 130, elastomeric bodyside liner 128, and any combination of other elastomeric components that in combination render a stretchable unitary chassis that does not compromise the structural integrity and absorbency of the absorbent article 132.

The training pant embodiment 100 may be of a style and configuration wherein the front and back ear portions 150, 152 have lateral sides that are brought together upon folding the chassis to form a pant-like structure having the waist opening 124 and leg openings 122. The lateral sides are bonded in a known manner so as to define side seams 126 (FIG. 3) of the pant structure. With this type of configuration, the pant 100 is pulled on by the wearer in a manner similar to underwear. Desirably, these seams 126 may be separable or tearable so that the pant 100 may be removed from the wearer by tearing the seams 126 and removing the article in a manner similar to a diaper. In an alternate embodiment, the front and back panel portions 150, 152 may be separable and re-attachable at the side seams 126. A fastening system, such as a hook-and-loop system, may be used to interconnect the first waist region 112 with the second waist region 114 to define the pant structure and hold the article on a wearer. Additional suitable releasable fastening systems are described in U.S. Pat. No. 6,231,557 B1 and International Application WO 00/35395, these references being incorporated herein by reference in their entirety for all purposes.

An article 100 according to the invention may also incorporate longitudinally extending containment flaps 159 disposed over the bodyside liner 128, as generally understood in the art and shown in FIGS. 3 and 4. The flaps 159 have longitudinal ends that are attached to the chassis 120 generally at the waistband portions 117, 111. In certain embodiments of the invention, the flaps 159 may comprise separate panels or sheets of material having an outboard lateral side that is attached to the chassis 120 desirably outboard of the underlying absorbent body 132. Referring to FIG. 3, the flaps 159 may be attached, for example, along the seam line 127. The flaps 159 have a laterally inboard “free” side 163 such that the guards essentially define a containment pocket along the lateral sides of the absorbent body 132. Referring to FIG. 4, the free sides 163 may incorporate flap elastics 137 along their longitudinal side, as is generally known in the art. The construction of such containment flaps 159 is well known and need not be described in detail. Suitable constructions and arrangements for the containment flaps 159 are described, for example, in U.S. Pat. No. 4,704,116, which is incorporated herein by reference for all purposes.

An article 100 according to the invention may also incorporate longitudinally extending leg elastics 133 (FIG. 4) to enhance the fit and leakage protection of the product. Suitable constructions and arrangements for leg elastics 133 are well known in the art and need not be described in detail here.

FIG. 4 shows a body facing plan view of a representative article 100, in this case a disposable diaper, in its generally flat-out, uncontracted state (i.e., with substantially all elastic-induced gathering and contraction removed). The diaper incorporates any manner of conventional securing or fastening device, such as hook or loop tabs 135 as illustrated. The tabs 135 may engage directly with the outer cover member or with corresponding loop or hook material provided on the outer cover member 130, as in known in the art. The components are attached or joined together by conventional suitable attachment methods such as adhesive bonds, sonic bonds, thermal bonds, pinning, stitching or any other attachment technique known in the art, as well as combinations thereof. For example, a uniform continuous layer of adhesive, a patterned layer of adhesive, a sprayed pattern of adhesive or an array of separate lines, swirls or spots of construction adhesive may be used to affix the various components.

The diaper 100 will typically include a porous, liquid permeable bodyside liner 128 overlying an absorbent body 132; a substantially liquid impermeable outer cover member 130; and the absorbent body 132 positioned and attached between the outer cover member 130 and bodyside liner 128. In certain embodiments, a surge layer 148 may be optionally located adjacent the absorbent body and attached, for example, by way of an adhesive. The diaper defines a longitudinal direction 24 and a lateral, transverse direction 22.

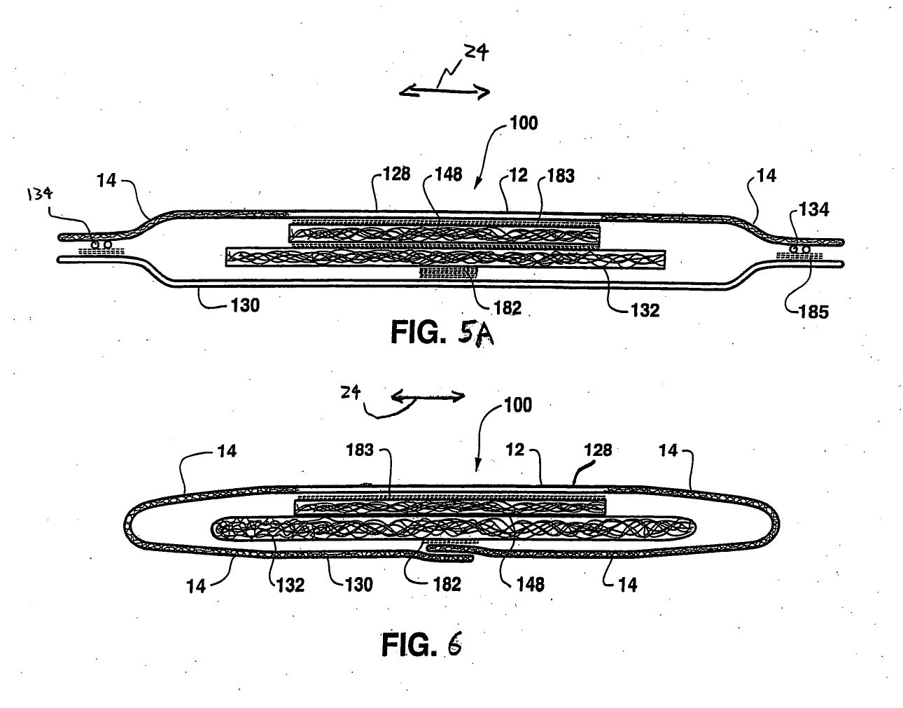

As depicted in FIG. 5A, the outer cover member 130 and bodyside liner 128 may be separate sheets joined at their respective longitudinal ends. Waist elastics 134 may be incorporated along the longitudinal end margins of the chassis 120 longitudinally outboard of the absorbent body 132 and are configured to draw and hold the chassis 120 against the torso of the wearer. The waist elastic members 134 are secured to the chassis 120 in an elastically contracted state so that in a normal under-strain condition, the waist elastic members 134 effectively contract against the wearer's body. The liner 128, outer cover 130, absorbent body 132, surge layer 148, and elastic members 134 may be assembled together into a variety of well-known absorbent article configurations.

In the embodiments illustrated in FIGS. 3 and 4, the waist elastics 134 are provided only partially across the front and back waistbands 117, 111. The waist elastics 134 may be composed of any suitable elastomeric material, such as an elastomeric film, an elastic foam, multiple elastic strands, an elastomeric fabric, and the like. Embodiments of waistband structures that may be utilized with articles 100 according to the invention are also described in U.S. Pat. Nos. 5,601,547; 5,500,063; 5,545,158; 6,358,350 B1; 6,336,921 B1; and 5,711,832, incorporated by reference to the extent consistent herewith.

In certain embodiments utilizing the composite material 10 according to the invention as described below, the composite elastomeric strips 14 may provide sufficient stretch properties to the chassis in the lateral direction such that the need for separately applied elasticized waistband structures is eliminated.

FIG. 5A is a schematic cross-sectional view of a disposable diaper 100 taken along the lines indicated in FIG. 4. (FIGS. 5-9 present views of longitudinally extending cross-sections of various disposable absorbent article embodiments.) In the embodiment depicted in FIG. 5A, the bodyside liner 128 is composed of the material 10 as described above in the various embodiments. In FIG. 5A (as well as FIGS. 6-9), the composite regions or strips 14 of the material 10 are shown with slight cross-hatching to represent that these portions are a multi-layer/composite elastic structure. The material 10 may be formed off-line and incorporated directly into the in-line manufacturing process of the absorbent article 100. Alternately, the material may be formed and conveyed directly into the in-line manufacturing of the articles 100. The base material 16 (FIG. 1) of the composite material is generally liquid permeable and may be any material suited for use as a bodyside liner. A surge layer 148 may be placed between the absorbent body 132 and center strip 12. It may be desired to adhere the entire portion of the strip 12 that overlies the absorbent body 132 to the absorbent body (or surge layer 148) with an adhesive 183. With this configuration, the capillary structure of the overlying region of the strip 12 is maintained even with longitudinal stretching of the composite strips 14. The composite elastomeric strips or regions 14 extend longitudinally outward from the center strip 12 to the longitudinal ends of the chassis 120 and are joined to the outer cover member 130, such as by thermal bonding and/or adhesive 185. The outer cover member 130 may be adhered to the absorbent body 132 with an adhesive, such as a transverse centerline adhesive 182. As mentioned, waist elastics 134 may be incorporated along the end seams between the outer cover member 130 and composite strips 14. In this configuration, the composite strips 14 provide a longitudinal stretchability to the bodyside liner 128. The composite strips 14 will stretch in the longitudinal direction 24 without imparting distorting tension to the non-extensible center strip 12 and underlying absorbent body 132. In this embodiment, it may be desired that the outer cover member 130 is also elastomeric in at least the longitudinal direction 24.

Various materials are available and known in the art for use as separate outer cover members 130. Constructions of the outer cover member 130 may comprise a woven or non-woven fibrous web layer which has been totally or partially constructed or treated to impart the desired levels of liquid impermeability to selected regions that are adjacent or proximate the absorbent body. Alternatively, a separate liquid impermeable material could be associated with the absorbent body 132. The outer cover may include a gas-permeable, nonwoven fabric layer laminated to a polymer film layer which may or may not be gas-permeable. Other examples of fibrous, cloth-like outer cover materials can comprise a stretch thinned or stretch thermal laminate material. Although the outer cover member 130 typically provides the outermost layer of the article, optionally the article may include a separate outer cover component member which is additional to the outer cover member.

As mentioned, the outer cover member 130 may be formed substantially from an elastomeric material. Alternately, the outer cover member may be formed from an extendable material that is non-elastomeric. The outer cover member 130 may, for example, be composed of a single layer, multiple layers, laminates, spunbond fabrics, films, meltblown fabrics, elastic netting, microporous web, bonded carded webs or foams comprised of elastomeric or polymeric materials. Elastomeric nonwoven laminate webs may include a nonwoven material joined to one or more gatherable nonwoven webs, films, or foams. Stretch Bonded Laminates (SBL), Neck Bonded Laminates (NBL), and Necked Stretch Bonded Laminates (NSBL) are examples of elastomeric composites. Examples of suitable materials are Spunbond-Meltblown fabrics, Spunbond-Meltblown-Spunbond fabrics, Spunbond fabrics, or laminates of such fabrics with films, foams, or other nonwoven webs. Elastomeric materials may include cast or blown films, foams, or meltblown fabrics composed of polyethylene, polypropylene, or polyolefin copolymers, as well as combinations thereof. The outer cover 130 may include materials that have elastomeric or extensible properties obtained through a mechanical process, printing process, heating process, or chemical treatment. For example, such materials may be apertured, creped, neck-stretched, heat activated, embossed, and micro-strained, and may be in the form of films, webs, and laminates.

An alternate embodiment of an absorbent article 100 in accordance with the invention is illustrated in FIG. 6, which is similar in many respects to the embodiment of FIG. 5A. With this embodiment, however, the elastomeric strips 14 have a sufficient length so as to wrap around the absorbent body 132 and attach to each other at some location generally “under” the absorbent body. Thus, the strips 14 essentially encase the absorbent body 132 and define the outer cover 130. The non-extensible center region 12 overlying the surge layer 148 is adhered generally entirely to the surge layer with an adhesive 183 such that the capillary structure of the region 12 is “set” and will generally not be affected by stretching of the end strips 14. The strips 14 are attached to the underside of the absorbent body 132 by a transverse strip of adhesive 182. With this configuration, the strips 14 define elastomeric end portions of the bodyside liner 128 and further define an elastomeric outer cover 130.

FIG. 8 illustrates an embodiment that is similar to the embodiment of FIG. 6 with the exception that a material 10 as illustrated in FIG. 2C is used. Here, the single composite end strip 14 has a sufficient length so as to fold under the absorbent body 132 and attach to the opposite longitudinal end of the center region 12 of non-extensible material. Thus, the single composite side strip 14 also defines the outer cover member 130.

It should be appreciated that different elastomeric properties can be achieved in different regions of the strips 14. Examples include two different materials disposed side-by-side (with or without partial overlap), two different materials overlapping, or a type of post treatment of part of an elastomeric layer, such as post-bonding a smaller region to generate different elastomeric properties in that sub-region. Referring to FIG. 1, for example, the composite material 10 may be formed by attaching two different strips of elastomeric material to each side of the non-extensible center region 12. In other words, the strip 18 would be defined by two adjacent strips (not shown), and strip 20 would be defined by two adjacent strips (not shown).

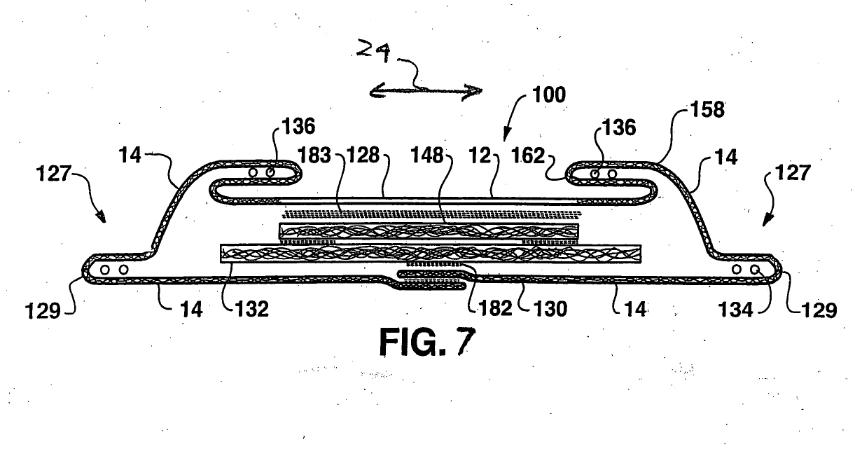

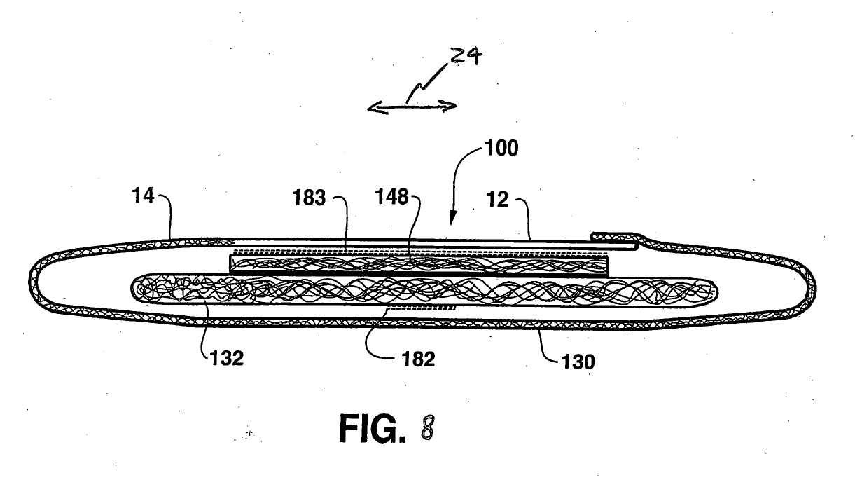

FIG. 7 illustrates another embodiment of an absorbent article 100 incorporating particular configurations of the composite material 10. The embodiment of FIG. 7 is similar in many respects to that of FIG. 6. The center region 12 has a sufficient length so as to overlie the surge layer 148 (or absorbent body 132 if a surge layer is not provided) and is attached to the surge layer 148 with an adhesive 183, as discussed above with respect to FIGS. 5 and 6. Furthermore, as with the embodiment of FIG. 6, the material 10 defines the bodyside liner 128 and the outer cover member 130, and provides desirable elastomeric stretch properties to these components. The base material 16 of the composite material 10 would again be selected to provide the desired characteristics of a bodyside liner, whereas the elastomeric materials 18 and 20 would be selected primarily to provide the desired characteristics of an outer cover member 130. However, in the embodiment of FIG. 7, waist elastics 134 are provided in the folded end margins. The waist elastic 134 can provide a snug fit to the waist regions of the wearer. Moreover, the embodiment of FIG. 7 depicts waist containment dams 158, defined by folded portions of the elastomeric composite regions 14. For example, the regions 14 may be folded in a Z-configuration as illustrated and incorporate elastic members 136 in the folded layers. A suitable adhesive may be used to attach the elastic members 136 and “set” the folded configuration. Such folded portions can be provided by the use of, for example, folding boards or folding knives in conjunction with the process representatively illustrated in FIG. 1; the use of folding boards and folding knives to impart folds to webs is well known in the art.

The embodiment of FIG. 9 is similar in many respects to that of FIG. 7 with the exception that the material 10 of FIG. 2C is used. In this embodiment, the single composite strip 14 is relatively long and is folded so as to define the waist containment dams 158 and the outer cover 130. The strip 14 is attached by any suitable means to the opposite longitudinal end of the center region 12 overlying the absorbent body 132.

The absorbent body 132 can be any structure or combination of components which are generally compressible, conformable, non-irritating to a wearer's skin, and capable of absorbing and retaining liquids and certain body wastes. For example, the body absorbent 132 may include an absorbent web material of cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. In a particular embodiment, the absorbent body 132 is a matrix of cellulosic fluff and superabsorbent hydrogel-forming particles. The cellulosic fluff may comprise a blend of wood pulp fluff. One preferred type of fluff is identified with the trade designation CR 1654, available from U.S. Alliance of Childersburg, Ala., USA, and is a bleached, highly absorbent wood pulp containing primarily soft wood fibers. The absorbent materials may be formed into a web structure by employing various conventional methods and techniques. For example, an absorbent web may be formed with a dry-forming technique, an air forming technique, a wet-forming technique, a foam-forming technique, or the like, as well as combinations thereof. Methods and apparatus for carrying out such techniques are well known in the art.

Superabsorbent materials are well known in the art and can be selected from natural, synthetic, and modified natural polymers and materials. The superabsorbent materials can be inorganic materials, such as silica gels, or organic compounds, such as crosslinked polymers. Typically, a superabsorbent material is capable of absorbing at least about 15 times its weight in liquid, and desirably is capable of absorbing more than about 25 times its weight in liquid. Suitable superabsorbent materials are readily available from various suppliers. For example, Favor 880 superabsorbent is available from Stockhausen GmbH of Germany; and Drytech 2035 is available from Dow Chemical Company, of Midland Mich., USA. In particular embodiments, the superabsorbent material is present in the absorbent web in an amount of from about 0 to about 90 weight percent based on total weight of the web. The web can have a density within the range of about 0.10 to about 0.35 grams per cubic centimeter.

After being formed or cut into a desired shape, the absorbent web material may be wrapped or encompassed by a suitable wrap that aids in maintaining the integrity and shape of the absorbent body 132.

The absorbent web material may also be a coform material. The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles, inorganic absorbent materials, treated polymeric staple fibers and the like. Any of a variety of synthetic polymers may be utilized as the melt-spun component of the coform material. For instance, in some embodiments, thermoplastic polymers can be utilized. Some examples of suitable thermoplastics that can be utilized include polyolefins, such as polyethylene, polypropylene, polybutylene and the like; polyamides; and polyesters. In one embodiment, the thermoplastic polymer is polypropylene. Some examples of such coform materials are disclosed in U.S. Pat. No. 4,100,324 to Anderson, et al.; U.S. Pat. No. 5,284,703 to Everhart, et al.; and U.S. Pat. No. 5,350,624 to Georger, et al.; which are incorporated herein in their entirety by reference thereto for all purposes.

The absorbent body 132 may include an elastomeric coform absorbent web material, for example as described in U.S. Pat. Nos. 4,663,220 and 4,741,949. In particular aspects, the elastomeric coform material can have an overall coform basis weight which is at least a minimum of about 50 g/m². The coform basis weight can alternatively be at least about 100 g/m² and can optionally be at least about 200 g/m² to provide improved performance. In addition, the coform basis weight can be not more than about 1200 g/m². Alternatively, the coform basis weight can be not more than about 900 g/m², and optionally, can be not more than about 800 g/m² to provide improved benefits. These values are important because they can in particular embodiments provide the absorbent body with desired stretchability and structural stability without excessively degrading the physical properties or the liquid-management functionalities of the absorbent body. Retention portions having excessively low proportions of elastomeric coform material may not be sufficiently stretchable. An absorbent web material having excessively large amounts of elastomeric coform materials can exhibit an excessive degradation of their absorbency functionalities, such as an excessive degradation of intake, distribution and/or retention properties.

Other examples of elastomeric absorbent bodies are described in U.S. Pat. No. 6,362,389 B1, incorporated herein by reference for all purposes.

The absorbent web material utilized in the absorbent body 132 is also selected so that the individual absorbent body possesses a particular individual total absorbency depending on the intended article of use. For example, for infant care products, the total absorbency can be within the range of about 200-900 grams of 0.9 wt % saline, and can typically be about 500 g of saline. For adult care products, the total absorbency can be within the range of about 400-2000 grams of saline, and can typically be about 1300 g of saline. For feminine care products, the total absorbency can be within the range of about 7-50 grams of menstrual fluid, and can typically be within the range of about 30-40 g of menstrual fluid.

As described, the absorbent body 132 may also include a surge management layer 148 which helps to decelerate and diffuse surges or gushes of liquid that may be rapidly introduced into the absorbent body of the article. Desirably, the surge management layer can rapidly accept and temporarily hold the liquid prior to releasing the liquid into the storage or retention portions of the absorbent body. The surge layer can be located below the bodyside liner layer 128. Alternatively, the surge layer may be located on the body facing surface of the bodyside liner 128. Examples of suitable surge management layers are described in U.S. Pat. No. 5,486,166; and U.S. Pat. No. 5,490,846. Other suitable surge management materials are described in U.S. Pat. No. 5,820,973. The entire disclosures of these patents are hereby incorporated by reference in their entirety for all purposes.

It should be understood that resort may be had to various other embodiments, modifications, and equivalents to the embodiments of the invention described herein which, after reading the description of the invention herein, may suggest themselves to those skilled in the art without departing from the scope and spirit of the present invention.

What is claimed is:

1. A disposable absorbent article, comprising:
a chassis defining front and back waist edges, opposite side edges extending between the front and back waist edges, a front waist region adjacent the front waist edge, a back waist region adjacent the back waist edge, a center region positioned longitudinally between the front and back waist regions, a longitudinal direction extending between and generally perpendicular to the front and back waist edges, and a lateral direction generally perpendicular to the longitudinal direction;
a garment-side outer cover;
a bodyside liner; and
an absorbent body sandwiched between the outer cover and the liner;
the liner comprising a base layer of fluid permeable material and further comprising a first strip of elastomeric material attached to the base layer material in the front waist region to define a first composite region and a second strip of elastomeric material attached to the base layer in the back waist region and longitudinally spaced from the first strip of elastomeric material to define a second composite region, the liner generally overlaying the absorbent body in the center region;
wherein the liner is substantially non-elastomeric in the center region, and wherein the liner is stretchable in the longitudinal direction in the front and back waist regions.

2. The disposable absorbent article of claim 1, wherein the base layer of fluid permeable material comprises a polyolefinic non-woven material.

3. The disposable absorbent article of claim 1, wherein the first and second strips of elastomeric material comprise an elastic film, the strips being laminated to the base layer material such that the composite regions are neck-bonded laminate regions.

4. The disposable absorbent article of claim 1, wherein the first and second strips of elastomeric materials comprise webs of elastomeric fibers.

5. The disposable absorbent article of claim 1, wherein the base layer material is tensioned in the lateral direction of the article before the first and second strips of elastomeric material are attached to the base layer.

6. The disposable absorbent article of claim 1, further comprising third and fourth strips of elastomeric material attached to the base layer material adjacent the side edges of the chassis to respectively define third and fourth composite regions, such that the center region of the base layer is bordered on its sides by the third and fourth strips.

7. The disposable absorbent article of claim 1, wherein the first and second composite regions are elastomeric in the longitudinal direction.

8. The disposable absorbent article of claim 1, wherein the first and second composite regions are elastomeric in both the longitudinal and lateral directions.

9. The disposable absorbent article of claim 1, wherein the base layer material comprises a creped material.

10. The disposable absorbent article of claim 1, wherein the bodyside liner is a separate component from the outer cover, the bodyside liner and the outer cover being attached generally along the waist and side edges of the chassis.

11. The disposable absorbent article of claim 10, wherein the outer cover is stretchable in the lateral direction.

12. The disposable absorbent article of claim 11, wherein the outer cover is elastomeric in the lateral direction.

13. The disposable absorbent article of claim 10, wherein the bodyside liner and the outer cover are generally coextensive around the entire periphery of the chassis.

14. The disposable absorbent article of claim 10, further comprising longitudinally extending containment flaps attached to the chassis atop the bodyside liner generally outboard of the absorbent body.

15. The disposable absorbent article of claim 10, wherein at least one of the composite regions includes a waist dam fold extending laterally between the side edges of the chassis to define a laterally extending waist containment dam.

16. The disposable absorbent article of claim 15, wherein an elastic member is enveloped within the waist dam fold.

17. The disposable absorbent article of claim 10, further comprising waist elastic members attached to the bodyside liner in the front and back waist regions of the chassis, such that front and back waist regions of the chassis are elastomeric in the lateral direction.

18. The disposable absorbent article of claim 1, wherein the first and second composite regions are folded toward a garment side of the chassis respectively at the front and back waist edges of the chassis to define front and back waist edge folds, the composite regions further longitudinally extending along the garment side of the chassis such that the composite regions constitute at least a portion of the outer cover.

19. The disposable absorbent article of claim 18, wherein at least one of the composite regions includes a waist dam fold extending laterally between the side edges of the chassis to define a laterally extending waist containment dam.

20. The disposable absorbent article of claim 19, wherein an elastic member is enveloped within the waist dam fold.

21. The disposable absorbent article of claim 18, wherein the first and second composite regions are attached to each other on the garment side of the chassis.

22. The disposable absorbent article of claim 21, wherein the first and second composite regions are attached to a garment side of the absorbent body.

23. The disposable absorbent article of claim 22, wherein bodyside portions of the composite regions are attached to garment-side portions of the composite regions along the opposite side edges of the chassis.

24. The disposable absorbent article of claim 18, further comprising a pair of front elastomeric side panels and a pair of back elastomeric side panels attached to the chassis along the opposite side edges thereof, wherein the front side panels are attached to the back side panels along side seams to define a pant-like structure.

25. The disposable absorbent article of claim 24, wherein the side seams are refastenable and comprise mechanical fasteners.

26. The disposable absorbent article of claim 18, further comprising waist elastic members attached to the first and second composite regions respectively within the front and back waist edge folds, such that front and back waist regions of the chassis are elastomeric in the transverse direction.

27. The disposable absorbent article of claim 18, wherein the first and second strips are integral with each other, such that the first strip, the second strip, and the outer cover form a single, continuous member.

28. The disposable absorbent article of claim 1, wherein the first and second strips of elastomeric material are sandwiched between the base layer of material and the absorbent body.

29. The disposable absorbent article of claim 1, wherein the first and second strips of elastomeric material are attached to a bodyside surface of the base layer of material, and wherein first and second strips of elastomeric material comprise a nonwoven layer.

30. The disposable absorbent article of claim 1, wherein the center region of the bodyside liner is extensible in the longitudinal direction of the article.

31. The disposable absorbent article of claim 30, wherein the base layer of material is a necked nonwoven material.

32. The disposable absorbent article of claim 30, wherein the base layer of material is an inherently extensible nonwoven material.

33. The disposable absorbent article of claim 1, wherein the article is a training pant or a disposable diaper.

34. A disposable absorbent article, comprising:
a chassis defining front and back waist edges, opposite side edges extending between the front and back waist edges, a front waist region adjacent the front waist edge, a back waist region adjacent the back waist edge, a center region positioned longitudinally between the front and back waist regions, a longitudinal direction extending between and generally perpendicular to the front and back waist edges, and a lateral direction generally perpendicular to the longitudinal direction;
a garment-side outer cover;
a bodyside liner; and
an absorbent body sandwiched between the outer cover and the liner;
the liner comprising a base layer of fluid permeable material and further comprising a first strip of elastomeric material attached to the base layer material in the front waist region to define a first composite region and a second strip of elastomeric material attached to the base layer in the back waist region and longitudinally spaced from the first strip of elastomeric material to define a second composite region, the liner generally overlaying the absorbent body in the center region;
wherein the liner is substantially non-elastomeric in the center region, and wherein the liner is elastomeric in the longitudinal direction in the front and back waist regions;
wherein the base layer of fluid permeable material comprises a poly-olefinic non-woven material;
the article further comprising longitudinally extending containment flaps attached to the chassis atop the bodyside liner generally outboard of the absorbent body;
wherein bodyside portions of the composite regions are attached to garment-side portions of the composite regions along the opposite side edges of the chassis.
